(19) United States
(12) Patent Application Publication
(10) Pub. No.: US 2025/0082902 A1
(43) Pub. Date: Mar. 13, 2025

(54) CATHETER HANDLE AND CATHETER WITH THE SAME

(71) Applicant: KANEKA CORPORATION, Osaka-shi, Osaka (JP)

(72) Inventor: Takuya KANEKO, Okaya-shi (JP)

(73) Assignee: KANEKA CORPORATION, Osaka-shi, Osaka (JP)

(21) Appl. No.: 18/718,111

(22) PCT Filed: Sep. 14, 2022
(86) PCT No.: PCT/JP2022/034433

§ 371 Date: Nov. 26, 2024

(30) Foreign Application Priority Data
Dec. 13, 2021 (JP) 2021-201976

(51) Int. Cl. A61M 25/01

(52) U.S. Cl. CPC A61M 25/0136

(57) ABSTRACT

A catheter handle (1) comprising: a rotary body (3) provided to be rotatable around an axial direction relative to the handle body (2), and having a first guiding part (19) extending in the axial direction; a shaft (4) disposed in an inner cavity of the rotary body (3), fixed to the handle body (2), having a first engagement part (11) extending spirally in one direction and a second engagement part (12) extending spirally in another direction, and a second guiding part (20) extending in the axial direction; a first slider (5), an inner surface of which is engaged with the first engagement part (11), having a fourth engagement part (14) on an outer surface thereof, which engages with the first guiding part (19); a second slider (6), an inner surface of which is engaged with the second engagement part (12), having a sixth engagement part (16) on an outer surface thereof, which engages with the first guiding part (19); a first bushing (7) rotatably connected to a distal side of the first slider (5), and engaged with the second guiding part (20), wherein a first wire (9) is fixed to; and a second bushing (8) rotatably connected to a distal side of the second slider (6), and engaged with the second guiding part (20), wherein a second wire (10) is fixed to.

### TECHNICAL FIELD

The present invention relates to a handle used for a catheter and a catheter equipped with the handle.

### BACKGROUND ART

A catheter usually comprises a tube for inserting into a body cavity such as a blood vessel, a digestive tract and an ureter tract, and a handle provided on a proximal side of the tube. Catheters in which a distal end portion of the tube can be bent by operating the handle provided on the proximal side are known. In such catheters, a wire is disposed in a lumen of the tube, a distal end portion of the wire is fixed to the distal end portion of the tube, a proximal end portion of the wire is connected to the handle, and the distal end portion of the tube can be bent by operating the handle. For example, in a catheter with two wires disposed in the lumen of the tube, the distal end portion of the tube can be bent to one direction by pulling one of the two wires proximally and to another direction by pulling the other thereof proximally by operating the handle.

As such catheter handles, for example, Patent Literature 1 discloses a catheter handle for controlling distal end deflection of a catheter, wherein the catheter includes first and second deflection wires, the handle comprising: a slide base including a slide compartment; an adjustment knob rotatably connected to the slide base and including a hole having an internal right thread and an internal left thread; a first slide located in the slide compartment, adapted to be coupled to the first deflection wire, and including an external right thread; and a second slide located in the slide compartment, adapted to be coupled to the second deflection wire, and including an external left thread; wherein the internal threads of the adjustment knob engage the external threads of the slides and rotation of the adjustment knob causes the slides to displace in opposite directions within the slide compartment. Patent Literature 2 discloses a catheter handle for operating a flexible distal end portion of a catheter tube, comprising: a housing, a rotary actuator provided to be rotatable around an axial direction of the housing; first and second sliders disposed inside the housing and being movable in the axial direction; a first anchor which is provided so as to be capable of contacting with and detaching from the first slider, to which a proximal portion of a first operating wire for deflecting the distal end portion of the tube in a first direction is fixed, and which is displaceable in the axial direction; a second anchor which is provided so as to be capable of contacting with and detaching from the second slider, to which a proximal portion of a second operating wire for deflecting the distal end portion of the tube in a second direction is fixed, and which is displaceable in the axial direction; and a feed mechanism that converts a rotational motion of the rotary actuator into a linear motion that moves the first and second sliders an equal distance in the axial direction in opposite direction to each other.

### CITATION LISTS

### Patent Literatures

PATENT LITERATURE 1
Japanese Unexamined Laid-open Patent Application Publication No. 2006-187606
PATENT LITERATURE 2
Japanese Unexamined Laid-open Patent Application Publication No. 2018-99480

### SUMMARY OF INVENTION

### Technical Problem

As described above, various catheter handles have been conventionally proposed, and an object of the present invention is to provide a catheter handle with a novel structure and a catheter equipped with the handle.

### Solution to Problem

The present invention includes the following catheter handle and catheter.
[1] A catheter handle for operating a tube of a catheter, comprising: a handle body; a rotary body, at least a part of which is disposed inside the handle body, having an inner cavity extending in an axial direction, provided to be rotatable around the axial direction relative to the handle body, and having a first guiding part extending in the axial direction on an inner surface of the inner cavity; a shaft disposed in the inner cavity of the rotary body, extending in the axial direction, fixed to the handle body, having a first portion and a second portion positioned proximal to the first portion with respect to the axial direction, having a first engagement part extending spirally on an outer surface of the first portion and a second engagement part extending spirally in an opposite direction to the first engagement part on an outer surface of the second portion, and having a second guiding part extending in the axial direction on outer surfaces of the first portion and the second portion; a first slider disposed on an outside of the shaft, having a third engagement part on an inner surface of the first slider, which engages with the first engagement part of the shaft, and having a fourth engagement part on an outer surface of the first slider, which engages with the first guiding part of the rotary body; a second slider disposed on the outside of the shaft, having a fifth engagement part on an inner surface of the second slider, which engages with the second engagement part of the shaft, and having a sixth engagement part on an outer surface of the second slider, which engages with the first guiding part of the rotary body; a first bushing disposed on the outside of the shaft, connected to a distal side of the first slider so as to be rotatable around the axial direction relative to the first slider, and having a seventh engagement part which engages with the second guiding part of the shaft; a second bushing disposed on the outside of the shaft, connected to a distal side of the second slider so as to be rotatable around the axial direction relative to the second slider, and having an eighth engagement part which engages with the second guiding part of the shaft; a first wire having a distal portion placed in a lumen of the tube and a proximal portion fixed to or engaged with the first bushing; and a second wire having a distal portion placed in a lumen of the tube and a proximal portion fixed to or engaged with the second bushing.
[2] The catheter handle according to [1] above, wherein one of the first engagement part and the third engagement part is a convex part and the other of the first engagement part and the third engagement part is a groove, and one of the second engagement part and the fourth engagement part is a convex part and the other of the second engagement part and the fourth engagement part is a groove.
[3] The catheter handle according to [1] or [2] above, wherein the first guiding part is a groove extending in the axial direction, and the fourth engagement part and the sixth engagement part are convex parts.
[4] The catheter handle according to any one of [1] to [3] above, wherein the second guiding part is a groove extending in the axial direction, and the seventh engagement part and the eighth engagement part are convex parts.
[5] The catheter handle according to any one of [1] to [4] above, wherein the proximal portion of the first wire is fixed to the first bushing, and the proximal portion of the second wire is fixed to the second bushing.
[6] The catheter handle according to any one of [1] to [4] above, wherein the first bushing has a first insertion opening which extends in the axial direction and through which the first wire is inserted, the first wire is provided with a stopper, which is larger than an aperture of the first insertion opening, on a proximal side of the first bushing, whereby the first wire is engaged with the first bushing, the second bushing has a second insertion opening which extends in the axial direction and through which the second wire is inserted, and the second wire is provided with a stopper, which is larger than an aperture of the second insertion opening, on a proximal side of the second bushing, whereby the second wire is engaged with the second bushing.
[7] A catheter comprising: a catheter handle according to any one of [1] to [6]above, and a tube provided on a distal side of the catheter handle.

### Advantageous Effects of Invention

According to the catheter handle and the catheter of the present invention, the tube provided on the distal side of the catheter handle can be operated by rotating the rotary body relative to the handle body.

### BRIEF DESCRIPTION OF DRAWINGS

FIG. 1 shows an overall view of a catheter provided with a catheter handle.

FIG. 2 shows a plan view of the internal structure of the catheter handle provided in the catheter shown in FIG. 1.

FIG. 3 shows a plan view of the internal structure of the catheter handle shown in FIG. 2 with a first slider, a second slider, a first bushing and a second bushing partially cut away along an axial direction.

FIG. 4 shows a plan view of the internal structure of the catheter handle shown in FIG. 2 with a shaft removed.

FIG. 5 shows a plan view of a shaft provided in the catheter shown in FIG. 2.

FIG. 6 shows an enlarged plan view of a first portion of a shaft, a first slider, a first bushing, and surroundings thereof in the internal structure of the catheter handle shown in FIG. 2.

FIG. 7 shows an enlarged plan view of a second portion of a shaft, a second slider, a second bushing, and surroundings thereof in the internal structure of the catheter handle shown in FIG. 2.

FIG. 8 shows a cross-sectional view taken along a line VIII-VIII of the catheter handle shown in FIG. 2.

FIG. 9 shows a cross-sectional view taken along a line IX-IX of the catheter handle shown in FIG. 2.

FIG. 10 shows a modified example of the catheter handle shown in FIG. 6.

FIG. 11 shows a cross-sectional view taken along a line XI-XI of the catheter handle shown in FIG. 10.

FIG. 12 shows a cross-sectional view taken along a line XII-XII of the catheter handle shown in FIG. 10.

### DESCRIPTION OF EMBODIMENTS

Hereinafter, a catheter handle and a catheter equipped with the handle of the present invention is specifically explained below based on the following embodiments; however, the present invention is not restricted by the embodiments described below of course, and can be certainly put into practice after appropriate modifications within in a range meeting the gist of the above and the below, all of which are included in the technical scope of the present invention. In the drawings, hatching or a reference sign for a member may be omitted for convenience, and in such a case, the description and other drawings should be referred to. In addition, sizes of various members in the drawings may differ from the actual sizes thereof, since priority is given to understanding the features of the present invention.

The overall structure of a catheter equipped with a catheter handle is explained with reference to FIG. 1. A catheter 31 according to an embodiment of the present invention comprises a catheter handle 1 (hereinafter may be referred to as “handle 1”) and a tube 32 provided on a distal side of the handle 1. The catheter 31 is used for, for example, treatment or examination by inserting the tube 32 into a body cavity such as a blood vessel and a digestive tract of a patient.

In the present invention, a proximal side of the catheter refers to a direction toward a user's hand with respect to an extending direction of the catheter, and a distal side refers to the opposite direction from the proximal side, that is, the direction toward a treatment target side.

The tube 32 has a flexible tubular structure, and can be made of, for example, synthetic resin such as polyolefin resins (e.g., polyethylene or polypropylene), polyamide resins (e.g., nylon), polyester resins (e.g., PET), aromatic polyetherketone resins (e.g., PEEK), polyether polyamide resins, polyurethane resins, polyimide resins and fluororesins (e.g., PTFE, PFA, ETFE), or metal such as stainless steel, carbon steel and nickel-titanium alloys. The metal material can also be used for a metal wire embedded within a synthetic resin tube. The length of the tube 32 in an axial direction (distal-proximal direction) is several times to several tens of times longer than the length of the handle 1 in the same direction, and is, for example, about 500 mm to 1200 mm. The outer diameter of the tube 32 may be, for example, approximately 0.6 mm to 5 mm.

The tube 32 has a lumen, and may have either a single lumen structure with one lumen or a multilumen structure with multiple lumens. The tube 32 may have a coaxial structure with a plurality of coaxial lumens. A wire for operating the tube 32 is disposed in the lumen of the tube 32. The wire is provided, for example, to control bending of a distal end portion of tube 32. In this case, a distal end portion of the wire is preferably fixed to the distal end portion of the tube 32, for example to a distal one-third portion of the tube 32. In the lumen of the tube 32, a conducting wire, an optical fiber, an endoscope or the like may be disposed and the tube 32 may have a lumen for inserting a guidewire or another treatment instrument, or a lumen for flowing a drug, a contrast agent or any fluid.

The handle 1 is provided on a proximal side of the tube 32, and when assembled as a catheter, a proximal end portion of the wire disposed in the lumen of the tube 32 is connected to the handle 1.

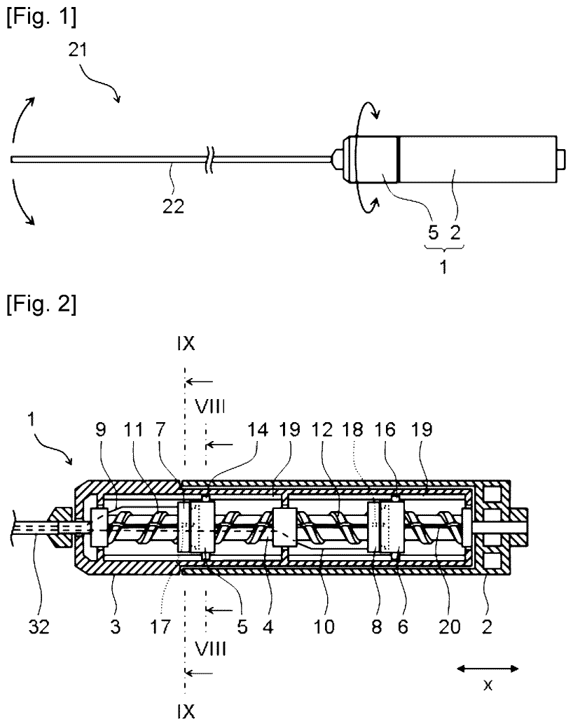

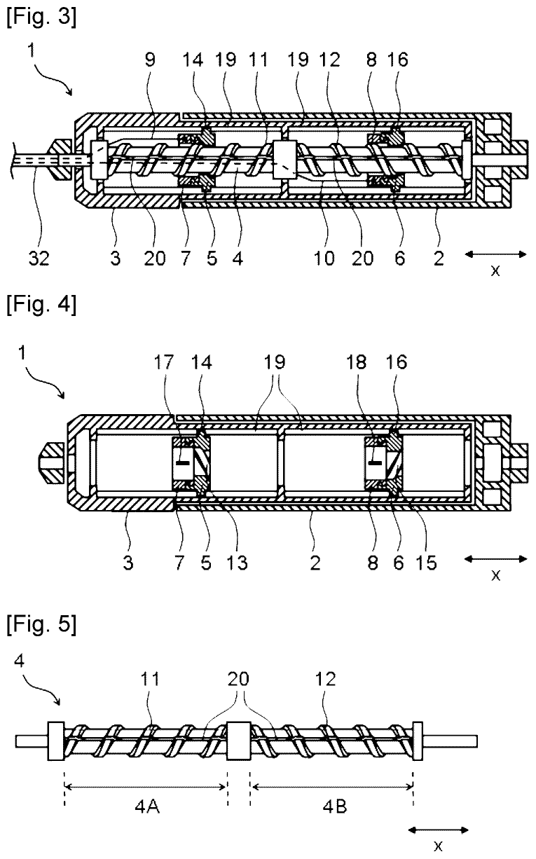

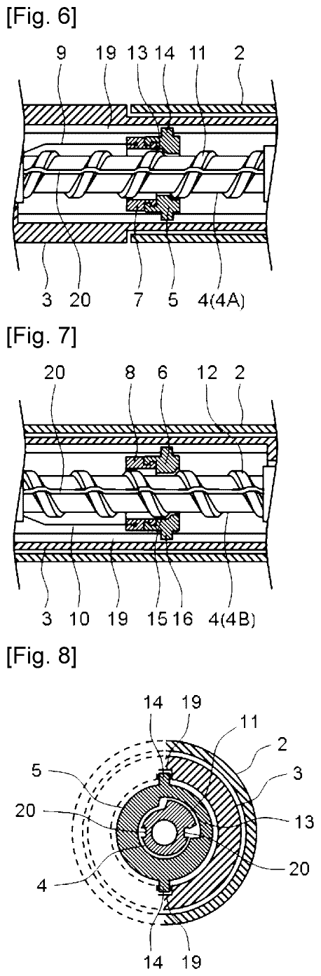

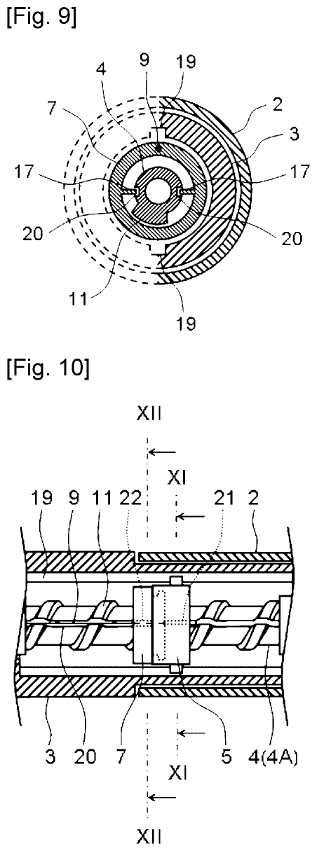

Details of the catheter handle is explained with reference to FIGS. 2 to 9. FIG. 2 shows a plan view of the internal structure of the catheter handle provided in the catheter shown in FIG. 1, FIGS. 3 to 7 show detailed views of the internal structure of the catheter handle shown in FIG. 2, FIG. 8 shows a cross-sectional view taken along a line VIII-VIII of the catheter handle shown in FIG. 2, and FIG. 9 shows a cross-sectional view taken along a line IX-IX of the catheter handle shown in FIG. 2. FIG. 2 shows a plan view of the catheter handle in which a handle body and a rotary body, which serve as a housing of the catheter handle, are cut away along an axial direction so that the internal structure can be seen. FIG. 3 shows a plan view of the internal structure of the catheter handle shown in FIG. 2 with a first slider, a second slider, a first bushing and a second bushing partially cut away along the axial direction. FIG. 4 shows a plan view of the internal structure of the catheter handle shown in FIG. 2 with a shaft removed, and FIG. 5 shows a plan view of the shaft provided in the catheter shown in FIG. 2. FIGS. 6 and 7 show an enlarged plan view of a first portion of the shaft, the first slider, the first bushing, and surroundings thereof and an enlarged plan view of a second portion of the shaft, the second slider, the second bushing, and surroundings thereof in the internal structure of the catheter handle shown in FIG. 2, respectively. In FIGS. 8 and 9, the cut away portion of the handle body and the rotary body in FIG. 2 is shown in dotted lines.

The handle 1 according to an embodiment of the present invention comprises: a handle body 2; a rotary body 3 disposed inside the handle body 2 and provided to be rotatable relative to the handle body 2; a shaft 4 disposed in the inner cavity of the rotary body 3 and fixed to the handle body 2. In the handle 1, the axial direction x corresponds to a direction in which a rotation axis of the rotary body 3 extends, and a proximal side and a distal side are defined as one side and the other side with respect to the axial direction x. Further, a radial direction is defined as a direction orthogonal to the axial direction x.

The handle body 2 has an inner cavity in which the rotary body 3 is disposed (see FIG. 4). The inner cavity of the handle body 2 preferably includes a cylindrical portion centered on the axial direction x, and the rotary body 3 is preferably disposed in the cylindrical portion of the inner cavity.

At least a part of the rotary body 3 is placed inside the handle body 2, and the rotary body 3 is provided to be rotatable around the axial direction x relative to the handle body 2. It is preferable that at least a part of an outer surface of the rotary body 3 is formed into a cylindrical shape, and at least a part of the cylindrical-shaped portion is preferably placed inside the handle body 2. The rotary body 3 is configured to be rotatably operable from the outside of the handle 1, and thus, it is preferable that the rotary body 3 is partially exposed to the outside of the handle body 2.

It is preferable that the handle body 2 is configured such that at least one of distal and proximal sides is open, and the rotary body 3 is inserted from the open distal or proximal side of the handle body 2, whereby the rotary body 3 is placed in the inner cavity of the handle body 2. In the drawings, the distal side of the handle body 2 is open, and the rotary body 3 is inserted into the inner cavity of the handle body 2 from the distal side of the handle body 2. In the handle 1, the handle body 2 and the rotary body 3 are exposed to the outside, and the handle 1 can be operated by, for example, holding the handle body 2 with one hand and the rotary body 3 with the other hand and rotating the rotary body 3 against the handle body 2.

The rotary body 3 has an inner cavity extending in the axial direction x, in which the shaft 4 is disposed so as to extend in the axial direction x (see FIG. 3). The shaft 4 is preferably arranged at a position that serves as a rotation axis of the rotary body 3. Thereby, rotational operation of the rotary body 3 is facilitated. The inner cavity of the rotary body 3 preferably includes a cylindrical portion centered on the axial direction x.

The tube 32 is preferably connected to a distal side of the shaft 4. A connection part between the shaft 4 and the tube 32 may be located in the inner cavity of the rotary body 3, may be located distal to the inner cavity of the rotary body 3, or may be located outside the handle 1. The shaft 4 may also be formed integrally with the tube 32.

The shaft 4 is fixed to the handle body 2 and is provided so as not to rotate or move relative to the handle body 2. To this end, it is preferable that an opening for passing the shaft 4 is provided at a proximal end of the rotary body 3, through which the shaft 4 extends proximally to the rotary body 3, and the shaft 4 is fixed to the handle body 2 outside the rotary body 3. The shaft 4 may be formed integrally with the handle body 2.

It is preferable that an opening for passing the shaft 4 is provided at a distal end of the rotary body 3, through which the shaft 4 extends distally to the rotary body 3. In the case where the connection part between the shaft 4 and the tube 32 is located in the inner cavity of the rotary body 3, it is preferable that the tube 32 is inserted into the opening provided at the distal end of the rotary body 3, and through this opening, the tube 32 extends proximally to the distal end of the rotary body 3.

The shaft 4 may be formed solid or may be formed hollow. It is preferable that the shaft 4 is formed hollow and a lumen of the shaft 4 communicates with the lumen of the tube 32. This allows a treatment instrument to be delivered to a treatment site or a drug or a contrast agent to be injected from the proximal side of the catheter through the lumen of the shaft 4 and the lumen of the tube 32. Examples of the treatment instrument include an electrode catheter, an ablation catheter, a mapping catheter, a balloon catheter, a microcatheter, forceps, a laser probe, a fiberscope, a high-frequency treatment instrument, an electrohydraulic impact fracture probe, and others. To allow insertion of such a treatment instrument, the shaft 4 preferably has a proximal opening on the outside of the handle 1. The shaft 4 may have a branch part, and the branch part of the shaft 4 may be provided with a switching cock. This makes it possible to separately provide, for example, an insertion port for a treatment instrument and an injection port for a drag or the like.

As shown in FIG. 5, the shaft 4 has a first portion 4A and a second portion 4B positioned proximal to the first portion 4A with respect to the axial direction x, a first engagement part 11 extending spirally is provided on the outer surface of the first portion 4A, and a second engagement part 12 extending spirally in the opposite direction to the first engagement part 11 is provided on the outer surface of the second portion 4B. The first engagement part 11 and the second engagement part 12 are provided on the outer surface of the shaft 4 so as to extend in a spiral shape with the axial direction x of the handle 1 as an axis, and are preferably provided on the outer surface of the shaft 4 formed in a cylindrical shape. The first engagement part 11 and the second engagement part 12 may be formed in a clockwise spiral shape or a counterclockwise spiral shape from the proximal side toward the distal side, and one of the first engagement part 11 and the second engagement part 12 is formed in a clockwise spiral shape and the other of those is formed in a counterclockwise spiral shape.

In the shaft 4, the length of the first portion 4A in the axial direction x and the length of the second portion 4B in the axial direction x are preferably approximately the same. The length of the first portion 4A in the axial direction x is, for example, preferably 0.8 times or more, more preferably 0.9 times or more, and preferably 1.2 times or less, more preferably 1.1 times or less of the length of the second portion 4B in the axial direction x. Pitch length (interval) of the spiral of the first engagement part 11 formed in the first portion 4A and pitch length (interval) of the spiral of the second engagement part 12 formed in the second portion 4B are preferably approximately the same. The pitch length of the spiral of the first engagement part 11 is, for example, preferably 0.8 times or more, more preferably 0.9 times or more, and preferably 1.2 times or less, more preferably 1.1 times or less of the pitch length of the spiral of the second engagement part 12.

The first engagement part 11 and the second engagement part 12 may be formed continuously or intermittently. The first engagement part 11 and the second engagement part 12 are preferably formed to extend continuously or intermittently in a spiral shape for one or more rounds, more preferably to extend for two or more rounds, and even more preferably to extend three or more rounds. In the drawings, the first engagement part 11 and the second engagement part 12 are formed intermittently by a second guiding part 20 formed on the outer surface of the shaft 4.

As shown in FIGS. 2 and 3, a first slider 5 and a second slider 6 are disposed on the outside of the shaft 4. The first slider 5 and the second slider 6 are disposed in the inner cavity of the rotary body 3, and are arranged on the outer side of the shaft 4 in the radial direction. It is preferable that the first slider 5 and the second slider 6 are formed in a cylindrical shape, and the shaft 4 is inserted through openings of the cylindrical first slider 5 and the cylindrical second slider 6.

The first slider 5 is disposed on the outside the first portion 4A of the shaft 4, and the second slider 6 is disposed on the outside the second portion 4B of the shaft 4. The first slider 5 and the second slider 6 are provided so as to be displaceable along the shaft 4 in the axial direction x. it is preferable that the first slider 5 has a length in the axial direction x shorter than the length of the first portion 4A of the shaft 4 in the axial direction x, and the second slider 6 has a length in the axial direction x shorter than the length of the second portion 4B of the shaft 4 in the axial direction x.

The first slider 5 and the second slider 6 are configured to displace in the axial direction x on the shaft 4 while rotating together with the rotary body 3 by rotating the rotary body 3 around the axial direction x. To this end, a first guiding part 19 extending in the axial direction x is provided on an inner surface of the inner cavity of the rotary body 3, a third engagement part 13 which engages with the first engagement part 11 of the shaft 4 is provided on an inner surface of the first slider 5, a fourth engagement part 14 which engages with the first guiding part 19 of the rotary body 3 is provided on an outer surface of the first slider 5, a fifth engagement part 15 which engages with the second engagement part 12 of the shaft 4 is provided on an inner surface of the second slider 6, and a sixth engagement part 16 which engages with the first guiding part 19 of the rotary body 3 is provided on an outer surface of the second slider 6 (see FIGS. 3, 4, and 6 to 8). The first slider 5 can be rotated together with the rotary body 3 by the fourth engagement part 14 engaging with the first guiding part 19 of the rotary body 3, and can be displaced in the axial direction x while rotating around the axial direction x by the third engagement part 13 engaging with the first engagement part 11 of the shaft 4. The second slider 6 can be rotated together with the rotary body 3 by the sixth engagement part 16 engaging with the first guiding part 19 of the rotary body 3, and can be displaced in the axial direction x while rotating around the axial direction x by the fifth engagement part 15 engaging with the second engagement part 12 of the shaft 4.

The third engagement part 13 is provided on the inner surface of the first slider 5 so as to extend spirally. Specifically, the third engagement part 13 is formed to extend in a spiral shape with the axial direction x of the handle 1 as an axis. The third engagement part 13 is preferably provided on the inner surface of the first slider 5 formed in a cylindrical shape. The third engagement part 13 may be formed continuously or intermittently.

The fifth engagement part 15 is provided on the inner surface of the second slider 6 so as to extend spirally. Specifically, the fifth engagement part 15 is formed to extend in a spiral shape with the axial direction x of the handle 1 as an axis. The fifth engagement part 15 is preferably provided on the inner surface of the second slider 6 formed in a cylindrical shape. However, as described above, since the second engagement part 12 is formed to extend spirally in the opposite direction to the first engagement part 11, the fifth engagement part 15 is formed to extend in a spiral shape in the opposite direction to the third engagement part 13. The fifth engagement part 15 may be formed continuously or intermittently.

The third engagement part 13 and the fifth engagement part 15 may be formed to extend continuously or intermittently in a spiral shape for one or more arounds or may be formed for less than one around. The third engagement part 13 and the fifth engagement part 15 may be formed, for example, so as to extend only half a round in a spiral shape. In the drawings, the third engagement part 13 and the fifth engagement part 15 are provided in a spiral shape extending less than one around.

The first engagement part 11 and the third engagement part 13 can be formed by a combination of a convex part and a groove. That is, it is preferable that one of the first engagement part 11 and the third engagement part 13 is a convex part and the other of those is a groove. In the case where the first engagement part 11 or the third engagement part 13 is formed as a groove, the groove is preferably formed to extend continuously in a spiral shape. The groove may be a bottomed groove or a through groove, and in view of ensuring the strength of the shaft 4 or the first slider 5, the groove is preferably a bottomed groove. Meanwhile, in the case where the first engagement part 11 or the third engagement part 13 is formed as a convex part, the convex part may be provided in a continuously extending spiral shape or in an intermittently extending spiral shape. In the drawings, the first engagement part 11 provided on the shaft 4 is formed as a convex part, and the third engagement part 13 provided on the first slider 5 is formed as a groove.

The second engagement part 12 and the fifth engagement part 15 can be formed by a combination of a convex part and a groove. That is, it is preferable that one of the second engagement part 12 and the fifth engagement part 15 is a convex part and the other of those is a groove. In the case where the second engagement part 12 or the fifth engagement part 15 is formed as a groove, the groove is preferably formed to extend continuously in a spiral shape. The groove may be a bottomed groove or a through groove, and in view of ensuring the strength of the shaft 4 or the second slider 6, the groove is preferably a bottomed groove. Meanwhile in the case where the second engagement part 12 or the fifth engagement part 15 is formed as a convex part, the convex part may be provided in a continuously extending spiral shape or in an intermittently extending spiral shape. In the drawings, the second engagement part 12 provided on the shaft 4 is formed as a convex part, and the fifth engagement part 15 provided on the second slider 6 is formed as a groove.

Since the first engagement part 11 is provided on the outer surface of the shaft 4 and the third engagement part 13 is provided on the inner surface of the first slider 5 as described above, a rotational movement of the first slider 5 around the axial direction x can be converted to a parallel movement in the axial direction x. In addition, since the second engagement part 12 is provided on the outer surface of the shaft 4 and the fifth engagement part 15 is provided on the inner surface of the second slider 6 as described above, a rotational movement of the second slider 6 around the axial direction x can be converted to a parallel movement in the axial direction x. And, to make the first slider 5 and the second slider 6 rotate around the axial direction x, the first guiding part 19 is provided on the inner surface of the rotary body 3, the fourth engagement part 14 is provided on the outer surface of the first slider 15, and the sixth engagement part 16 is provided on the outer surface of the second slider 6.

The first guiding part 19 is provided on the inner surface of the inner cavity of the rotary body 3 so as to extend in the axial direction x. The first guiding part 19 is provided at a position overlapping with the first portion 4A and the second portion 4B of the shaft 4 in the axial direction x, and is provided to face the first portion 4A and the second portion 4B of the shaft 4. In the first guiding part 19, a part facing the first portion 4A and a part facing the second portion 4B may be provided continuously, or the part facing the first portion 4A and the part facing the second portion 4B may be provided separately.

The first guiding part 19 can be formed as a convex part or a groove. In the case where the first guiding part 19 is formed as a convex part, the first guiding part 19 may be provided to extend continuously or intermittently in the axial direction x at the part facing the first portion 4A or the part facing the second portion 4B. In the case where the first guiding part 19 is formed as a groove, the first guiding part 19 is preferably provided to extend continuously in the axial direction x at the part facing the first portion 4A or the part facing the second portion 4B. In the drawings, the first guiding part 19 is formed as a groove.

It is preferable that the length of the part of the first guiding part 19 that faces the first portion 4A in the axial direction x is longer than the length of the first slider 5 in the axial direction x. In view of enabling the first slider 5 to move over a wide range in the axial direction x at the first portion 4A of the shaft 4, the length, with respect to the axial direction x, of the part of the first guiding part 19 that faces the first portion 4A in the axial direction x is preferably 0.7 times or more, more preferably 0.8 times or more, and even more preferably 0.9 times or more the length of the first portion 4A of the shaft 4 in the axial direction x.

It is preferable that the length of the part of the first guiding part 19 that faces the second portion 4B in the axial direction x is longer than the length of the second slider 6 in the axial direction x. In view of enabling the second slider 6 to move over a wide range in the axial direction x in the second portion 4B of the shaft 4, the length, with respect to the axial direction x, of the part of the first guiding part 19 that faces the second portion 4B is preferably 0.7 times or more, more preferably 0.8 times or more, and even more preferably 0.9 times or more the length of the second portion 4B of the shaft 4 in the axial direction x.

The fourth engagement part 14 of the first slider 5 is provided on the outer surface of the first slider 5 so as to engage with the first guiding part 19. The first guiding part 19 (specifically, the part of the first guiding part 19 that faces the first portion 4A) and the fourth engagement part 14 can be formed by a combination of a convex part and a groove. That is, it is preferable that one of the first guiding part 19 and the fourth engagement part 14 is a convex part and the other of those is a groove. The groove may be a bottomed groove or a through groove, and in view of ensuring the strength of the rotary body 3 or the first slider 5, the groove is preferably a bottomed groove. In the drawings, the first guiding part 19 is formed as a groove, and the fourth engagement part 14 is formed as a convex part. In the case where the fourth engagement part 14 is formed as a convex part, the fourth engagement part 14 of the convex part may be formed as a mere protrusion, or may be formed to extend continuously or intermittently in the axial direction x. Meanwhile, although not shown in the drawings, in the case where the fourth engagement part 14 is formed as a groove, the fourth engagement part 14 of a groove is preferably formed continuously so as to extend across the first slider 5 in the axial direction x.

The first guiding part 19 (specifically, the part of the first guiding part 19 that faces the first portion 4A) and the fourth engagement part 14 may be provided at only one location or at two or more locations, viewed from the proximal or distal side of the rotary body 3 or the first slider 5. However, in view of smoothly rotating the first slider 5 with the rotation of the rotary body 3, the first guiding part 19 and the fourth engagement part 14 are preferably provided at two or more locations, viewed from the proximal or distal side of the rotary body 3 or the first slider 5. In this case, it is preferable that the first guiding part 19 and the fourth engagement part 14 are arranged approximately evenly in the circumferential direction (i.e., a direction surrounding the axial direction x) on the inner surface of the rotary body 3 or the outer surface of the first slider 5.

The sixth engagement part 16 of the second slider 6 is provided on the outer surface of the second slider 6 so as to engage with the first guiding part 19. The first guiding part 19 (specifically, the part of the first guiding part 19 that faces the second portion 4B) and the sixth engagement part 16 can be formed by a combination of a convex part and a groove. That is, it is preferable that one of the first guiding part 19 and the sixth engagement part 16 is a convex part and the other of those is a groove. The groove may be a bottomed groove or a through groove, and in view of ensuring the strength of the rotary body 3 or the second slider 6, the groove is preferably a bottomed groove. In the drawings, the first guiding part 19 is formed as a groove, and the sixth engagement part 16 is formed as a convex part. In the case where the sixth engagement part 16 is formed as a convex part, the sixth engagement part 16 of the convex part may be formed as a mere protrusion, or may be formed to extend continuously or intermittently in the axial direction x. Meanwhile, although not shown in the drawings, in the case where the sixth engagement part 16 is formed as a groove, the sixth engagement part 16 of a groove is preferably formed continuously so as to extend across the second slider 6 in the axial direction x.

The first guiding part 19 (specifically, the part of the first guiding part 19 that faces the second portion 4B) and the sixth engagement part 16 may be provided at only one location or at two or more locations, viewed from the proximal or distal side of the rotary body 3 or the second slider 6. However, in view of smoothly rotating the second slider 6 with the rotation of the rotary body 3, the first guiding part 19 and the sixth engagement part 16 are preferably provided at two or more locations, viewed from the proximal or distal side of the rotary body 3 or the second slider 6. In this case, it is preferable that the first guiding part 19 and the sixth engagement part 16 are arranged approximately evenly in the circumferential direction (i.e., a direction surrounding the axial direction x) on the inner surface of the rotary body 3 or the outer surface of the second slider 6.

The part of the first guiding part 19 that faces the first portion 4A and the part of the first guiding part 19 that faces the second portion 4B may be provided at overlapping locations to each other or may be provided at different locations from each other, viewed from the proximal or distal side of the rotary body 3. Preferably, the first guiding part 19 is formed in the former manner.

It is preferable that the first guiding part 19 is a groove extending in the axial direction x, and the fourth engagement part 14 and the sixth engagement part 16 are convex parts. In the drawings, the first guiding part 19, the fourth engagement part 14 and the sixth engagement part 16 are formed in this manner. Thereby, the size of the handle 1 in the radial direction can be made compact. Further, when rotating the rotary body 3, a first bushing 7 and a second bushing 8, which are described later, are prevented from being caught by the first guiding part 19. Therefore, the first bushing 7 is easily rotated smoothly around the axial direction x relative to the first slider 5, and the second bushing 8 is easily rotated smoothly around the axial direction x relative to the second slider 6.

Since the first slider 5 and the second slider 6 are configured as described above, the rotation of the rotary body 3 around the axial direction x causes the displacement of the first slider 5 and the second slider 6 in opposite directions to each other with respect to the axial direction x. In the handle 1 shown in the drawings, when rotating the rotary body 3 clockwise as viewed from the proximal side, the first slider 5 moves proximally on the shaft 4 while rotating clockwise, and the second slider 6 moves distally on the shaft 4 while rotating clockwise. Meanwhile, when rotating the rotary body 3 counterclockwise as viewed from the proximal side, the first slider 5 moves distally on the shaft 4 while rotating counterclockwise, and the second slider 6 moves proximally on the shaft 4 while rotating counterclockwise.

As shown in detail in FIG. 6, a first bushing 7, which is connected to a distal side of the first slider 5 so as to be rotatable around the axial direction x relative to the first slider 5, is provided on the outside of the shaft 4. As shown in detail in FIG. 7, a second bushing 8, which is connected to a distal side of the second slider 6 so as to be rotatable around the axial direction x relative to the second slider 6, is provided. The first bushing 7 is provided for fixing or engaging a proximal portion of a first wire 9, a distal portion of which is placed in the tube 32 and the proximal portion of which is placed in the handle 1, and the second bushing 8 is provided for fixing or engaging a proximal portion of a second wire 10, a distal portion of which is placed in the tube 32 and the proximal portion of which is placed in the handle 1.

The first bushing 7 and the second bushing 8 are disposed in the inner cavity of the rotary body 3, and are arranged on the outer side of the shaft 4 in the radial direction. The first bushing 7 and the second bushing 8 are formed in a cylindrical shape, and the shaft 4 is inserted through openings of the cylindrical first bushing 7 and the cylindrical second bushing 8. The first bushing 7 is disposed on the outside of the first portion 4A of the shaft 4, and the second bushing 8 is disposed on the outside of the second portion 4B of the shaft 4.

The first bushing 7 is configured so as to be displaced in the axial direction x together with the first slider 5 but not to rotate around the axial direction x together with the rotary body 3. Therefore, the first bushing 7 is rotatably connected to the first slider 5 about the axial direction x. For example, the first bushing 7 is fitted to the first slider 5 so as to be rotatable around the axial direction x relative to the first slider 5. Furthermore, a second guiding part 20 extending in the axial direction x is provided on the outer surface of the shaft 4, and a seventh engagement part 17 which engages with the second guiding part 20 of the shaft 4 is provided on an inner surface of the first bushing 7. (see FIGS. 4, 6 and 9).

The second bushing 8 is configured so as to be displaced in the axial direction x together with the second slider 6 but not to rotate around the axial direction x together with the rotary body 3. Therefore, the second bushing 8 is rotatably connected to the second slider 6 about the axial direction x. For example, the second bushing 8 is fitted to the second slider 6 so as to be rotatable around the axial direction x. Furthermore, the second guiding part 20 extending in the axial direction x is provided on the outer surface of the shaft 4, and an eighth engagement part 18 which engages with the second guiding part 20 of the shaft 4 is provided on an inner surface of the second bushing 8. (see FIGS. 4 and 7).

The second guiding part 20 is provided on the first portion 4A and the second portion 4B of the shaft 4. The second guiding part 20 may be provided continuously from the first portion 4A to the second portion 4B, or may be provided separately in the first portion 4A and the second portion 4B.

The second guiding part 20 can be formed as a convex part or a groove. In the case where the second guiding part 20 is formed as a convex part, the second guiding part 20 is provided to extend in the axial direction x, and is preferably interrupted at a point overlapping the first engagement part 11 in the first portion 4A and is preferably interrupted at a point overlapping the second engagement part 12 in the second portion 4B. Thereby, the second guiding part 20 can be prevented from interfering with the first engagement part 11 and the second engagement part 12 on the outer surface of the shaft 4. In the case where the second guiding part 20 is formed as a groove, the second guiding part 20 is preferably provided to extend continuously in the first portion 4A, and is preferably provided to extend continuously in the second portion 4B. In the drawings, the second guiding part 20 is formed as a groove.

It is preferable that the length of the second guiding part 20 in the axial direction x at the first portion 4A is longer than the length of the first bushing 7 in the axial direction x. In view of enabling the first bushing 7 to move over a wide range in the axial direction x at the first portion 4A of the shaft 4, the length of the second guiding part 20 in the axial direction x at the first portion 4A is, for example, preferably 0.7 times or more, more preferably 0.8 times or more, and even more preferably 0.9 times or more the length of the first portion 4A of the shaft 4 in the axial direction x.

It is preferable that the length of the second guiding part 20 in the axial direction x at the second portion 4B is longer than the length of the second bushing 8 in the axial direction x. In view of enabling the second bushing 8 to move over a wide range in the axial direction x at the second portion 4B of the shaft 4, the length of the second guiding part 20 in the axial direction x at the second portion 4B is, for example, preferably 0.7 times or more, more preferably 0.8 times or more, and even more preferably 0.9 times or more the length of the second portion 4B of the shaft 4 in the axial direction x.

The seventh engagement part 17 of the first bushing 7 is provided on the inner surface of the first bushing 7 so as to engage with the second guiding part 20 at the first portion 4A of the shaft 4. The second guiding part 20 (specifically, the second guiding part 20 at the first portion 4A) and the seventh engagement part 17 can be formed by a combination of a convex part and a groove. That is, it is preferable that one of the second guiding part 20 and the seventh engagement part 17 is a convex part and the other of those is a groove. The groove may be a bottomed groove or a through groove, and in view of ensuring the strength of the shaft 4 or the first bushing 7, the groove is preferably a bottomed groove. In the drawings, the second guiding part 20 is formed as a groove, and the seventh engagement part 17 is formed as a convex part. In the case where the seventh engagement part 17 is formed as a convex part, the seventh engagement part 17 of the convex part may be formed as a mere protrusion, or may be formed to extend continuously or intermittently in the axial direction x. Meanwhile, although not shown in the drawings, in the case where the seventh engagement part 17 is formed as a groove, the seventh engagement part 17 of a groove is preferably formed continuously so as to extend across the first bushing 7 in the axial direction x on the inner surface of the first bushing 7.

The second guiding part 20 (specifically, the second guiding part 20 at the first portion 4A) and the seventh engagement part 17 may be provided at only one location or at two or more locations, viewed from the proximal or distal side of the shaft 4 or the first bushing 7. However, in view of allowing the first bushing 7 to move smoothly on the shaft 4 in the axial direction x, the second guiding part 20 and the seventh engagement part 17 are preferably provided at two or more locations, viewed from the proximal or distal side of the shaft 4 or the first bushing 7. In this case, it is preferable that the second guiding part 20 and the seventh engagement part 17 are arranged approximately evenly in the circumferential direction (i.e., a direction surrounding the axial direction x) on the outer surface of the shaft 4 or the inner surface of the first bushing 7.

The eighth engagement part 18 of the second bushing 8 is provided on the inner surface of the second bushing 8 so as to engage with the second guiding part 20 at the second portion 4B of the shaft 4. The second guiding part 20 (specifically, the second guiding part 20 at the second portion 4B) and the eighth engagement part 18 can be formed by a combination of a convex part and a groove. That is, it is preferable that one of the second guiding part 20 and the eighth engagement part 18 is a convex part and the other of those is a groove.

The groove may be a bottomed groove or a through groove, and in view of ensuring the strength of the shaft 4 or the second bushing 8, the groove is preferably a bottomed groove. In the drawings, the second guiding part 20 is formed as a groove, and the eighth engagement part 18 is formed as a convex part. In the case where the eighth engagement part 18 is formed as a convex part, the eighth engagement part 18 of the convex part may be formed as a mere protrusion, or may be formed to extend continuously or intermittently in the axial direction x. Meanwhile, although not shown in the drawings, in the case where the eighth engagement part 18 is formed as a groove, the eighth engagement part 18 of a groove is preferably formed continuously so as to extend across the second bushing 8 in the axial direction x on the inner surface of the second bushing 8.

The second guiding part 20 (specifically, the second guiding part 20 at the second portion 4B) and the eighth engagement part 18 may be provided at only one location or at two or more locations, viewed from the proximal or distal side of the shaft 4 or the second bushing 8. However, in view of allowing the second bushing 8 to move smoothly on the shaft 4 in the axial direction x, the second guiding part 20 and the eighth engagement part 18 are preferably provided at two or more locations, viewed from the proximal or distal side of the shaft 4 or the second bushing 8. In this case, it is preferable that the second guiding part 20 and the eighth engagement part 18 are arranged approximately evenly in the circumferential direction (i.e., a direction surrounding the axial direction x) on the outer surface of the shaft 4 or the inner surface of the second bushing 8.

The second guiding part 20 formed in the first portion 4A and the second guiding part 20 formed in the second portion 4B may be provided at overlapping locations to each other or may be provided at different locations from each other, viewed from the proximal or distal side of the shaft 4. Preferably, the second guiding part 20 is formed in the former manner.

It is preferable that the second guiding part 20 is a groove extending in the axial direction x, and the seventh engagement part 17 and the eighth engagement part 18 are convex parts. In the drawings, the second guiding part 20, the seventh engagement part 17 and the eighth engagement part 18 are formed in this manner. As a result, it becomes easier to form the second guiding part 20 so as not to interfere with the first engagement part 11 and the second engagement part 12 on the outer surface of the shaft 4. Further, the first slider 5 and the second slider 6 are prevented from being caught by the second guiding part 20, and it becomes easy that the first slider 5 and the second slider 6 are smoothly rotated spirally around the axial direction x relative to the shaft 4.

The first bushing 7 and the second bushing 8 are not provided with an engagement part that engages with the first engagement part 11 or the second engagement part 12 of the shaft 4, and are not provided with an engagement part that engages with the first guiding part 19 of the rotary body 3. Meanwhile, the first slider 5 and the second slider 6 are not provided with an engagement part that engages with the second guiding part 20 of the shaft 4.

Since the first bushing 7 and the second bushing 8 are configured as described above, the first bushing 7 and the second bushing 8 can be displaced in the axial direction x without rotating around the axial direction x when rotating the rotary body 3. As a result, the first wire 9 and the second wire 10 that are fixed to or engaged with the first bushing 7 or the second bushing 8 can be displaced in the axial direction x inside the handle body 2 without wrapping around the shaft 4.

A proximal end portion of the first wire 9 is fixed to or engaged with the first bushing 7. A distal portion of the first wire 9 is placed in the lumen of the tube 32. Preferably, the distal end portion of the first wire 9 is fixed to the distal end portion of the tube 32. In this case, the distal end portion of the tube 32 can be bent in one direction when operating the handle 1 to pull the first wire 9 proximally. The proximal portion of the first wire 9 is preferably provided in the inner cavity of the rotary body 3 so as to extend from the lumen of the shaft 4 or the tube 32 to the outside thereof. The proximal portion of the first wire 9 preferably extends from the lumen of the shaft 4 or the tube 32 to the outside thereof on a distal side of the first bushing 7 with the first bushing 7 displaced at a most distal side.

A proximal end portion of the second wire 10 is fixed to or engaged with the second bushing 8. A distal portion of the second wire 10 is placed in the lumen of the tube 32. Preferably, the distal end portion of the second wire 10 is fixed to the distal end portion of the tube 32. In this case, the distal end portion of the tube 32 can be bent in another direction when operating the handle 1 to pull the second wire 10 proximally. The proximal portion of the second wire 10 is preferably provided in the inner cavity of the rotary body 3 so as to extend from the lumen of the shaft 4 or the tube 32 to the outside thereof. The proximal portion of the second wire 10 preferably extends from the lumen of the shaft 4 or the tube 32 to the outside thereof on a distal side of the second bushing 8 and a proximal side of the first bushing 7 with the second bushing 8 displaced at a most distal side.

The distal end portion of the first wire 9 can be defined, for example, as a portion within 100 mm proximally from a distal end of the first wire 9, and the distal end portion of the second wire 10 can be defined, for example, as a portion within 100 mm proximally from a distal end of the second wire 10. The distal end portion of the tube 32 can be defined, for example, as a portion within 100 mm proximally from a distal end of the tube 32.

For the first wire 9 and the second wire 10, a metal wire such as stainless steel, carbon steel, and nickel-titanium alloy, or a resin wire (i.e., fiber material) made of synthetic resin such as polyamide resin (e.g., nylon), polyolefin resin (e.g., polyethylene and polypropylene), polyester resin (e.g., PET), aromatic polyetherketone resin (e.g., PEEK), polyimide resin, aromatic polyamide resin (e.g., aramid), and fluororesin (e.g., PTFE, PFA, FEP, ETFE) can be used. These metal and resin wires may have a monofilament structure or a multifilament structure. The first wire 9 and the second wire 10 may be covered with a tubular body formed of a coiled metal or synthetic resin. The diameters of the first wire 9 and the second wire 10 can be, for example, about 100 μm to 1500 μm.

In FIGS. 6 and 7, examples in which the first wire 9 is fixed to the first bushing 7 and the second wire 10 is fixed to the second bushing 8 are shown. In the case where the first wire 9 is fixed to the first bushing 7, the first wire 9 is connected to the first bushing 7 from a distal side of the first bushing 7 and is fixed to the first bushing 7. The first wire 9 may be fixed to a distal surface of the first bushing 7, may be fixed to a proximal surface of the first bushing 7, or may be fixed to an interior of the first bushing 7. In the case where the second wire 10 is fixed to the second bushing 8, the second wire 10 is connected to the second bushing 8 from a distal side of the second bushing 8 and is fixed to the second bushing 8. The second wire 10 may be fixed to a distal surface of the second bushing 8, may be fixed to a proximal surface of the second bushing 8, or may be fixed to an interior of the second bushing 8. Examples of methods of fixing the first wire 9 to the first bushing 7 and fixing the second wire 10 to the second bushing 8 include, for example, bonding with an adhesive, welding, fitting, caulking, screwing, and others. In this case, the first wire 9 can be displaced proximally or distally together with the first bushing 7 by the displacement of the first bushing 7 proximally or distally. The second wire 10 can be displaced proximally or distally together with the second bushing 8 by the displacement of the second bushing 8 proximally or distally.

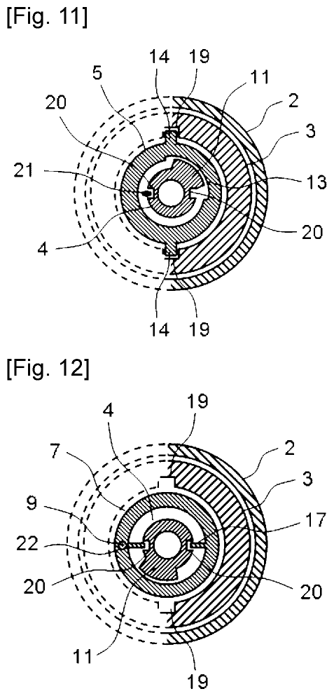

In FIGS. 10 to 12, an example in which the first wire 9 is engaged with the first bushing 7 is shown. FIG. 10 shows a modified example of the internal structure, that is, a modified example of the internal structure of the first portion of the shaft, the first slider, the first bushing and the surroundings thereof, of the catheter handle shown in FIG. 6, FIG. 11 shows a cross-sectional view taken along a line XI-XI of the catheter handle shown in FIG. 10, and FIG. 12 shows a cross-sectional view taken along XII-XII of the catheter handle shown in FIG. 10.

In the case where the first wire 9 is engaged with the first bushing 7, the first wire 9 is preferably engaged with a proximal surface of the first bushing 7. For example, it is preferable that the first wire 9 is provided with a stopper 21 and the stopper 21 is engaged with the proximal surface of the first bushing 7 so as to be capable of contacting with and detaching from the proximal surface of the first bushing 7. It is preferable that a first insertion opening 22 for the first wire 9 extending in the axial direction x is formed in the first bushing 7, the first wire 9 is inserted through the first insertion opening 22, and the stopper 21 which is larger than an aperture of the first insertion opening 22 is provided on the first wire 9 on the proximal side of the first bushing 7. In this case, due to the displacement of the first bushing 7 to the proximal side, the stopper 21 provided on the first wire 9 comes into contact with the proximal surface of the first bushing 7, the first wire 9 is pulled proximally by the proximal surface of the first bushing 7, and the first wire 9 can be displaced proximally together with the first bushing 7. When the first bushing 7 is displaced distally, the proximal portion of the first wire 9 can be displaced distally.

In the case where the second guiding part 20 of the shaft 4 is formed as a groove, the stopper 21 may be placed in the groove of the second guiding part 20. In this case, when the first bushing 7 is displaced distally, the first wire 9 is released from the force of being pulled proximally by the first bushing 7, and restorative force of the first wire 9 placed in the tube 32 (that is, a distal displacement of the first wire 9 placed in the lumen of the tube 32) allows the proximal portion of the first wire 9 to displace distally. The stopper 21 of the first wire 9 may extend proximally beyond the proximal end of the first slider 5 through the groove of the second guiding part 20, or may be pulled proximally by an elastic member such as a spring or rubber.

Although not shown in the drawings, a recessed part may be formed on the distal surface of the first bushing 7, and the stopper of the first wire 9 may be placed in this recessed part. In this case, when the first bushing 7 is displaced distally, the stopper provided on the first wire 9 comes into contact with the distal surface of the first slider 5 and the first wire 9 is pushed distally by the distal surface of the first slider 5, whereby the proximal portion of the first wire 9 can be displaced distally.

In the case where the second wire 10 is engaged with the second bushing 8, the second wire 10 is preferably engaged with a proximal surface of the second bushing 8. In this case as well, the second wire 10 may be provided with a stopper, and the stopper may be configured to be engaged with the proximal surface of the second bushing 8 so as to be capable of contacting with and detaching from the proximal surface of the second bushing 8. It is preferable that a second insertion opening for the second wire 10 extending in the axial direction x is formed in the second bushing 8, the second wire 10 is inserted through the second insertion opening, and the stopper which is larger than an aperture of the second insertion opening is provided on the second wire 10 on the proximal side of the second bushing 8. In this case, due to the displacement of the second bushing 8 to the proximal side, the stopper provided on the second wire 10 comes into contact with the proximal surface of the second bushing 8, the second wire 10 is pulled proximally by the proximal surface of the second bushing 8, and the second wire 10 can be displaced proximally together with the second bushing 8. When the second bushing 8 is displaced distally, the proximal portion of the second wire 10 can be displaced distally.

In the case where the second guiding part 20 of the shaft 4 is formed as a groove, the stopper of the second wire 10 may be placed in the groove of the second guiding part 20. In this case, when the second bushing 8 is displaced distally, the second wire 10 is released from the force of being pulled proximally by the second bushing 8, and restorative force of the second wire 10 placed in the tube 32 (that is, a distal displacement of the second wire 10 placed in the lumen of the tube 32) allows the proximal portion of the second wire 10 to displace distally. The stopper of the second wire 10 may extend proximally beyond the proximal end of the second slider 6 through the groove of the second guiding part 20, or may be pulled proximally by an elastic member such as a spring or rubber.

A recessed part may be formed on the distal surface of the second bushing 8, and the stopper of the second wire 10 may be placed in this recessed part. In this case, when the second bushing 8 is displaced distally, the stopper provided on the second wire 10 comes into contact with the distal surface of the second slider 6, and the second wire 10 is pushed distally by the distal surface of the second slider 6, whereby the proximal portion of the second wire 10 can be displaced distally.

In the case where the second guiding part 20 of the shaft 4 is formed as a groove, the first wire 9 and the second wire 10 may be placed in the groove of the second guiding part 20 of the shaft 4 in the inner cavity of the rotary body 3. This makes it easy to stably displace the proximal portion of the first wire 9 and the proximal portion of the second wire 10 in the axial direction x in the inner cavity of the rotary body 3.

The catheter handle of the present invention has been described above, but the catheter handle of the present invention is not limited to one which controls bending of the distal end portion of the tube, as long as it displaces the first wire and the second wire distally or proximally in the lumen of the tube. For example, the first wire and/or the second wire may be moved in and out of a distal end of the tube by operating the catheter handle. Alternatively, a treatment tool such as a knife or a snare may be attached to the distal end portion of the first wire or the distal end portion of the second wire and the treatment tool attached to the distal end portion of the first wire or the distal end portion of the second wire can be inserted or withdrawn from the distal end of the tube by operating the catheter handle.

This application claims priority to Japanese Patent Application No. 2021-201976 filed on Dec. 13, 2021. All of the contents of the Japanese Patent Application No. 2021-201976 filed on Dec. 13, 2021 is incorporated by reference herein.

### REFERENCE SIGNS LIST

1: catheter handle
2: handle body
3: rotary body
4: shaft, 4A: first portion, 4B: second portion
5: first slider
6: second slider
7: first bushing
8: second bushing
9: first wire
10: second wire
11: first engagement part
12: second engagement part
13: third engagement part
14: fourth engagement part
15: fifth engagement part
16: sixth engagement part
17: seventh engagement part
18: eighth engagement part
19: first guiding part
20: second guiding part
21: stopper
22: first insertion opening
31: catheter
32: tube

1. A catheter handle for operating a tube of a catheter, comprising:
a handle body;
a rotary body, at least a part of which is disposed inside the handle body, having an inner cavity extending in an axial direction, provided to be rotatable around the axial direction relative to the handle body, and having a first guiding part extending in the axial direction on an inner surface of the inner cavity;
a shaft disposed in the inner cavity of the rotary body, extending in the axial direction, fixed to the handle body, having a first portion and a second portion positioned proximal to the first portion with respect to the axial direction, having a first engagement part extending spirally on an outer surface of the first portion and a second engagement part extending spirally in an opposite direction to the first engagement part on an outer surface of the second portion, and having a second guiding part extending in the axial direction on outer surfaces of the first portion and the second portion;
a first slider disposed on an outside of the shaft, having a third engagement part on an inner surface of the first slider, which engages with the first engagement part of the shaft, and having a fourth engagement part on an outer surface of the first slider, which engages with the first guiding part of the rotary body;
a second slider disposed on the outside of the shaft, having a fifth engagement part on an inner surface of the second slider, which engages with the second engagement part of the shaft, and having a sixth engagement part on an outer surface of the second slider, which engages with the first guiding part of the rotary body;
a first bushing disposed on the outside of the shaft, connected to a distal side of the first slider so as to be rotatable around the axial direction relative to the first slider, and having a seventh engagement part which engages with the second guiding part of the shaft;
a second bushing disposed on the outside of the shaft, connected to a distal side of the second slider so as to be rotatable around the axial direction relative to the second slider, and having an eighth engagement part which engages with the second guiding part of the shaft;
a first wire having a distal portion placed in a lumen of the tube and a proximal portion fixed to or engaged with the first bushing; and
a second wire having a distal portion placed in a lumen of the tube and a proximal portion fixed to or engaged with the second bushing.

2. The catheter handle according to claim 1, wherein
one of the first engagement part and the third engagement part is a convex part and the other of the first engagement part and the third engagement part is a groove, and
one of the second engagement part and the fourth engagement part is a convex part and the other of the second engagement part and the fourth engagement part is a groove.

3. The catheter handle according to claim 1, wherein
the first guiding part is a groove extending in the axial direction, and the fourth engagement part and the sixth engagement part are convex parts.

4. The catheter handle according to claim 1, wherein
the second guiding part is a groove extending in the axial direction, and the seventh engagement part and the eighth engagement part are convex parts.

5. The catheter handle according to claim 1, wherein
the proximal portion of the first wire is fixed to the first bushing, and the proximal portion of the second wire is fixed to the second bushing.

6. The catheter handle according to claim 1, wherein
the first bushing has a first insertion opening which extends in the axial direction and through which the first wire is inserted,
the first wire is provided with a stopper, which is larger than an aperture of the first insertion opening, on a proximal side of the first bushing, whereby the first wire is engaged with the first bushing,
the second bushing has a second insertion opening which extends in the axial direction and through which the second wire is inserted, and
the second wire is provided with a stopper, which is larger than an aperture of the second insertion opening, on a proximal side of the second bushing, whereby the second wire is engaged with the second bushing.

7. A catheter comprising:
a catheter handle according to any one of claims 1 to 6; and
a tube provided on a distal side of the catheter handle.
